(19) United States
(12) Patent Application Publication
(10) Pub. No.: US 2025/0312521 A1
(43) Pub. Date: Oct. 9, 2025

(54) METHOD FOR MANAGING BREAST MILK EXPRESSION

(71) Applicant: MEDELA HOLDING AG, Baar (CH)

(72) Inventors: Danielle Prime, Bateau Bay (AU); Leon Mitoulas, Stirling Wa (AU); Brian H. Silver, Woodstock, IL (US)

(21) Appl. No.: 18/628,035

(22) Filed: Apr. 5, 2024

(51) Int. Cl. A61M 1/06

(52) U.S. Cl. CPC A61M 1/0693

(57) ABSTRACT

A method for managing breast milk expression including providing an expected milk yield over time; expressing milk and measuring the expressed milk yield at a given time; comparing the measured milk yield at the given time with the expected milk yield at the given time and generating an alarm if the measured milk yield is lower than the expected yield.

### FIELD OF THE INVENTION

The present invention relates generally to a method for managing breast milk expression, and more particular relates generally to a software, a method for operating a breast pump and a breast pump with managed breast milk expression.

### BACKGROUND

Breast pumps for use by nursing mothers are well known. They allow the nursing woman to express the breast milk as necessary or convenient, and further provide collection of the breast milk for later use. For some mothers, breast pumps may be a necessity, such as when the child has sucking problems, or if the mother has problems with excessive or deficient milk production, or soreness, deformation, or injury of the mammilla. Electrically driven breast pumps are commonplace. They typically include an aggregate and a controller for controlling the aggregate as well as an energy supply connected to the aggregate via power cord or battery. The aggregate of the breast pump can be any unit, which transforms the electric energy into suction or motion eventually generating suction. This in general also applies to the breast pump which may be used in connection with the present invention. Advantages of this type of pump are convenience, ready controllability and regulation of the vacuum, and in some instances the ability to pump both breasts at once.

The controller is usually adapted to generate the vacuum (negative pressure) to be applied at the breast geared to a particular sequence, or curve, of negative pressure increase (i.e., increasing suction), and then release. This is often aimed at reproducing in some sense the sucking action of a healthy baby during mature lactation, after the milk supply has been established; see for example WO 00/57934. This may be achieved by operating the breast pump with at least one pattern, which may comprise a single or various sequences. Each sequence is defined by a specific cycle rate and a vacuum level; see US2010217182 A1, US2016082164 A1. The sequences in particular applied in the first days after birth of the baby are to stimulate a high and constant yield, which is usually developed about one or two weeks after birth.

Nowadays technology has developed to control the electrically driven motorized breast pumps with a pumping performance, which is realistically imitating the sucking of a real baby. In this respect, the breast pump of the invention and the method of the invention for operating the breast pump may implement the state of the art routines and/or pattern and/or sequences.

### Problem

Tests showed that a certain percentage of mothers may start to produce milk as expected but then start to reduce milk production compared to the expected yield.

Often milk production reduction occurs about eight days after birth of the baby; since milk production in the long term depends on the milk expression close to baby birth, e.g. at day 14 from the baby birth, a reduction of milk production immediately after or close to the baby birth negatively affects milk production in the long term, e.g. also after months after the baby birth. In addition, milk production reduction often also appears about 12 weeks after the baby birth; also in this case, such a reduction affects the milk production in the long term.

Milk reduction often results in a much lower yield than originally expected after the milk reduction has occurred and in particular in the long term, so that milk produced by the mother may not suffice for feeding the baby, in particular after weeks or months after his or her birth.

### SUMMARY OF THE INVENTION

The present invention, as defined by the appended claims, provides an improved method for managing the expression of milk for a baby and may also initiate an enhanced yield of milk.

According to a first aspect, the present invention provides a method for managing breast milk expression comprising the steps of providing an expected milk yield over time, preferably with reference to the baby birth, expressing milk and measuring the expressed milk yield at a given time, preferably with reference to the baby birth, and comparing a measured yield at a given time with the expected yield at that time and generating an alarm if the expected yield is lower that the measured yield.

An alarm is an information e.g. in form of a signal, that is generated to be made available to the mother and/or to other stakeholders (e.g. father, healthcare physician, doctor, etc.) and or to be used in the process itself, e.g. to trigger other actions, as explained in the following.

Outputting of an alarm may depend on the observation of a yield deficit, which yield deficit will be explained further below. The alarm can be generated with a delay with respect to the given time. This way for example if the measured milk yield is lower than the expected milk yield, the alarm is not generated immediately but e.g. after few hours or one day or more days, so that there is the possibility for the mother to naturally recover milk production.

The milk yield has a tolerance value, so that the alarm is generated when the measured milk yield is lower than the expected milk yield tolerance value. The tolerance value may be 10% or 5% or 3% less than the expected milk yield of each day or may be 10% or 5% or 3% less than the expected milk yield of a given day after the baby birth, e.g. at day 6, or also 10% or 5% or 3% less than the average of the expected milk yield of two or more given days.

The method can be adapted for use with a motor-driven breast pump, preferably having an aggregate and a controller.

The measured yield may be measured by determining the volume or weight of the milk within a container. The measured yield may likewise be measured by appropriate means provided by the pump, which means may communicate with a controller of the pump. The means may comprise a sensor, in particular a sensor within a breast pump. The measured yield may be measured by a flow sensor or a scale or by monitoring pressure profiles within the breast pump and/or monitoring movement of at least one component within the pump liked e.g. a membrane which is driven by the aggregate. Various pieces of information can be used to measure or estimate yield over time. Part of this information may be contributed by the breast pump, in particular the controller thereof. The controller may measure in the above meaning and/or calculate the yield over time or may receive data of a measurement allowing to measure and/or calculate the yield over time.

The expected yield may be taken from literature and/or may be implemented by the user of the breast pump, e.g. based on personal deliberations or studies or experience. For example, the controller may store the expected milk yield over a time in its memory.

The expected yield or target yield is compared with a measured yield to assess whether the measured yield is in accordance with what can be expected or was assumed to be expected. Naturally, a certain range below the expected or target yield may not necessarily be regarded to be critical/indicate an initial decline of the yield as the yield over time increment may in any event vary to a certain extent over the days of using the breast pump.

However, any abnormality, which indicates an early reduction of milk production compared with the expected behavior, will be calculated to indicate a yield deficit. This yield deficit is to be understood as an early indicator for a reduced milk production. In case breast-feeding mothers start to produce less milk than expected, such incident usually occurs at around day eight and/or 12 weeks after birth of the baby. While the reasons may be many like stress, return to work, or specific ways of using the breast pump, the incident per se may lead to a steady decline in yield. Milk production decrease affects milk production in the long term, i.e. in case milk production decreases, e.g. at around day eight and/or 12 weeks after baby birth, milk production stays at lower levels that expected also after such a decline occurs. Thus and according to an embodiment of the present invention, the date of birth, possibly including the hour of the birth of said date, is entered into the software or control of the breast pump.

The present invention aims to give early notification of such negative incident, which will eventually lead to a lack of available milk for the baby, so that measures can be taken to stimulate milk production, so that milk production goes back to the expected amounts.

The method may be implemented in any way, e.g. it may be implemented by a software running on a smartphone or tablet or computer that has stored the expected milk yield and that is periodically updated with the measured milk yield to generate a warning in case the measured milk yield is lower than the expected milk yield.

The method may also be implemented by the controller of a breast pump, where the controller runs a software that implements the method. The controller may for example output a warning to the user. This warning may be an acoustic, visual, haptic, or tactile warning. An acoustic or visual warning may be given for example by an external pump, which is connected to a breast shield by tubing and may be placed on a table or sofa during use. The warning may likewise be visual information showing the yield path for the expected yield on one hand in relation to a path or single measurement result of the measured yield.

The warning may trigger or incentivize countermeasures. Any measure may manually or automatically be applied which is known to potentially increase milk yield of a lactating mother. Adaption of the operation of the pump by the user or manually massaging the breast may be a measure of choice. In addition or alternatively, the alarm may incentivize the mother to adopt the way of life in particular to reduce the stress level or nutrition to foster milk yield.

An alternative measure could be the adoption of at least one operational parameter of the breast pump, which operational parameter will increase yield.

The operational parameter, which will be changed by the controller in case of a yield deficit, may include cycle rate, vacuum level, pull suction and/or release suction. Vacuum level is the average vacuum provided by one cycle. One cycle has a vacuum profile that comprises a pull suction and reducing the pull suction to a release suction and raising the suction pressure to reach the pull suction again. Suction pressure in the present description is a negative pressure. The higher the pull suction, the higher the absolute value of the pressure and the lower the pressure value as the same is negative. A cycle may have at least one period of constant pressure e.g. at the release suction value and/or the pull suction value and/or a constant pressure in-between the pull suction and the release suction.

Apart from changing the parameters defining a single cycle, the breast pump may be activated to apply a massage to the breast. Such massage may be applied for example by an in-bra breast pump having an aggregate within a housing, which housing is worn within the bra of the user. The aggregate or other element within the housing may vibrate to apply such massaging effect of the in-bra breast pump.

According to a preferred embodiment, the controller is adapted to apply a breast pumping session with a pattern of sequences. The pattern comprises at least one stimulation sequence for stimulating the breast having a rapid cycle rate and providing a moderate vacuum level and at least one expression sequence having a lower cycle rate and providing a stronger vacuum level, wherein the lower cycle rate is lower than the rapid cycle rate. In case of a yield deficit the controller changes at least one of the cycle rate, the vacuum level and the duration of at least one of the sequences in order to increase yield.

For this to achieve, methods and routines can be applied as described previously e.g. in US2010217182 A1, US2016082164 A1 the disclosure thereof is hereby incorporated herein by reference.

The present invention likewise proposes a software and a breast pump, which are capable of performing the inventive method.

The software can for example be executed by at least one programmable processor of a smartphone or tablet, or computer or other electronic device.

The breast pump can include an aggregate and a controller, which controller is adapted to control the aggregate. The controller can have at least one programmable processor that executes a software implementing the method on the present disclosure.

Furthermore, the controller may be adapted to receive or calculate the yield of expressed milk due to the pumping operation (measured yield), for which the breast pump may have at least one sensor for sensing milk flow and/or a logic within the controller to deduce information on the yield of expressed milk due to the performance of the pump or the operational settings of the pump.

The controller may also be adapted to store an inputted target yield over time and/or to estimate or calculate an expected yield over time based on the yield outputted by the controller (expected yield).

The controller is furthermore adapted to calculate the yield deficit by comparing the measured yield with the expected yield. Furthermore, the controller is adapted to output a warning and/or change at least one operational parameter of the breast pump for increasing yield upon notifying a yield deficit.

In connection with the description of the invention, the following terms shall be understood as follows:

“Sequence” is a sequence of multiple, on a regular basis identical cycles.

“Cycle” is a cyclic element for example of a sequence. Each “cycle” provides a vacuum profile consisting of a pull suction, reducing the pull suction to a release suction and raising the suction pressure to reach pull suction again, possible with at least one period of constant pressure at release suction and/or at pull suction and/or a suction level between release suction and pull suction.

“Vacuum level” can be understood as the average vacuum provided by one cycle. The “vacuum level” is defined by two pressure values, one being the pressure of value of pull suction, the other being the pressure value of release suction.

“Pull suction” is the suction pressure, i.e. negative pressure.

“Release suction” is the release pressure and may be positive, negative or ambient pressure.

A “sucking pattern” is specified by a specific cycle rate and a vacuum level.

“Cycle rate” provides information on the cycles per time increment.

### BRIEF DESCRIPTION OF THE DRAWINGS

These and other advantages of the invention will be further understood upon consideration of the following detailed description of certain embodiments, taken in conjunction with the drawings, in which:

FIG. 1 is an illustration of a breast pump assembly for use in accordance with one embodiment of the present invention;

FIG. 2 is a first graph that shows the average daily milk output from a mother's breast over the first fourteen days after birth of the baby;

FIG. 3 is a second graph that shows the average daily milk output from a mother's breast over the first fourteen days after birth of the baby; and

FIG. 4 is a diagram, in the form of a plot of suction pressure versus time, exemplifying process parameters of the breast pump.

### DETAILED DESCRIPTION

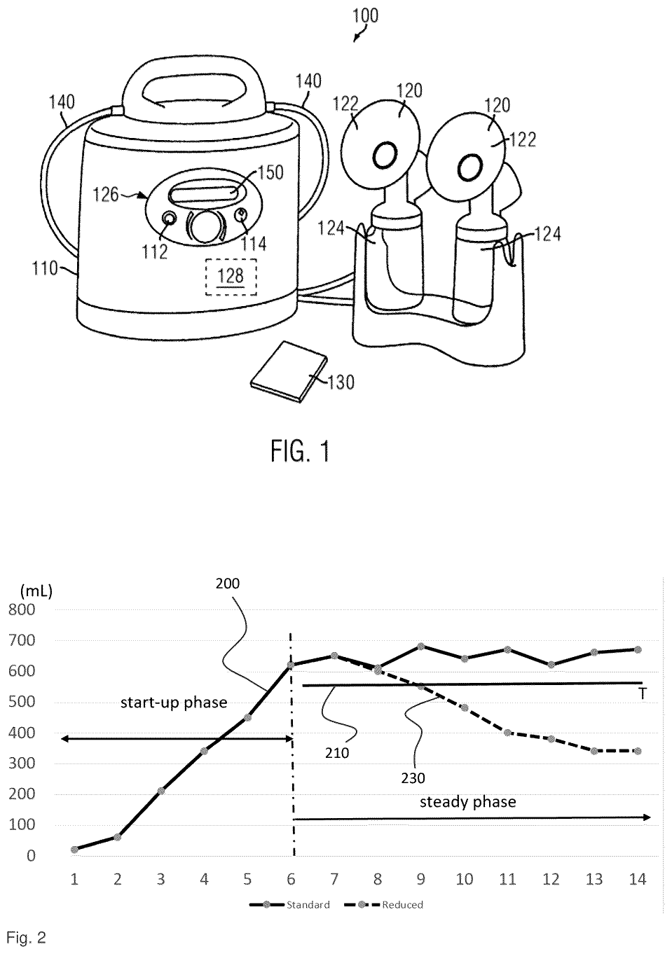

FIG. 1 is an illustration of a breast pump assembly in accordance with one embodiment of the present invention. That breast pump is generally described in U.S. Pat. No. 6,547,756 B or US2010217182 A1, US2016082164 A1, reference thereto can be made for salient details of this breast pump. While the invention has been described with reference to this kind of programmable breast pump and with respect to premature babies, it can be used or adapted for use with other motorized pumps, and aspects are considered adaptable to full-term babies including breast pumps wearable inside a bra, as disclosed in WO 2022/268998 A1.

As shown in FIG. 1, the breast pump assembly 100 includes a breast pump 110, a plurality of the breast shield and container assemblies 120. Power may be provided to the breast pump apparatus 110 through standard current via a power cord, either a battery, or some other appropriate power supply.

The breast pump 110 may be either a double or a single pump. The single pump extracts milk from one breast at a time, and the double pump can be used to extract milk from both breasts at the same time. The breast pump 110 is attached to each of the plurality of breast shield and container assemblies 120 with a tube 140. Each of the plurality of breast shield and container assemblies 120 comprises a breast shield 122 and a container 124. The container 124 is used to store the pumped milk.

The breast pump 110 utilizes a controller 126 for controlling an aggregate 128. The controller 126 or microprocessor-based system is provided with user input interface 130. The user input interface 130 is shown as a program card 130 in the attached figures, but in different examples it can be of any other different type. The controller 126 is provided within a housing of the breast pump 110 to control the aggregate 128 for providing the suction sequences of the suction pattern described hereinafter.

To extract breast milk from a mother, breast shields 122 are placed and centered over a mother's nipples. The breast pump apparatus 110 may be turned on by a user pressing a first button 112. The breast pump 110 may display instructions to the user via the interface 150. The instructions may ask the user to start the program. If the mother wants to start the program, the mother may press a second button 114. The interface 150 may then show instructions and/or graphics that let the mother know that the program is starting.

The breast pump may operate a preset pattern comprised of non-repeating suction sequences. Alternatively, the breast pump may operate a preset pattern comprised of repeating suction sequences that appear to be non-repeating to the user, due to the overall length of the pumping session such that the sequences seem to be occurring at unpredictable times in the pattern with extended pauses between some of the sequences. An extended pause is a pause that is approximately greater than about five seconds in duration. Some of the pauses may be of shorter duration, however, but still of a length that yields a noticeable hiatus to the mother.

The sequences comprise at least one stimulation sequence and one expression sequence, and may additionally comprise further stimulation sequences and/or expression sequences as described in US2010217182 A1, US2016082164 A1.

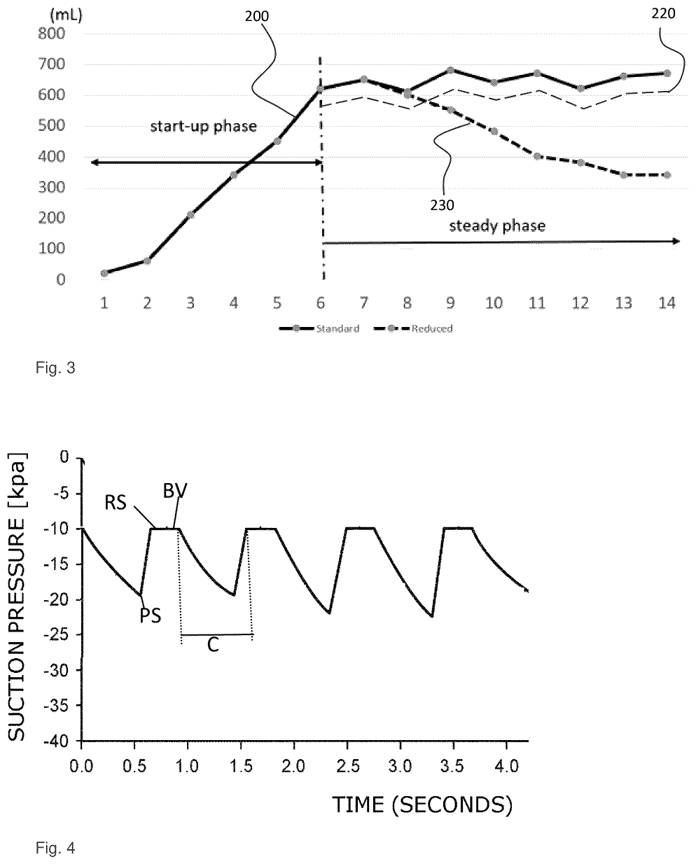

FIGS. 2 and 3 exemplify yields in form of average daily milk output in mL obtainable by a breast pump as described above. The expected or standard yield is the milk yield produced by mothers that do not show milk yield reduction. This can be calculated as an average of milk expressed by many mothers and is also available in literature.

Mothers start milk production after birth of the baby, because the baby sucking milk from the mother's breast stimulates milk production and expression. For this reason, after baby birth milk production rises till day 6 after birth of the baby, when milk production stabilizes (reference 200 in FIGS. 2 and 3).

Therefore, in a standard development of yield and during the initial start-up phase in the first six days after birth of the baby, yield of expressed milk will rise. In the example, at the sixth day an expected yield over time of about 620 m is obtained. For the standard path and during the steady phase following the start-up phase, this yield will not fall below a tolerance value.

The tolerance value may in a first example (FIG. 2) be identified as the horizontal bold line 210 in FIG. 2. The tolerance value may be about 10% or 5% or 3% less than the expected yield at day 6 or day 7 or may be about 10% or 5% or 3% less than the average of the expected yield at day 6 and day 7.

Alternatively, the tolerance value may vary every day and so each day it can be about 10% or 5% or 3% less than the expected milk yield of that day; this is shown by the dashed line 220 in FIG. 3.

In case the mother has no milk yield problems, she will continue to produce milk according to the expected milk yield line (solid line).

The hashed line 230 exemplifies an incident of milk production decrease, which e.g. occurs at day eight after baby birth with a first measured yield of expressed milk per day less than for the standard path. In the example, at day nine, the yield falls below the tolerance value (in the exampled defined by line 210), which will cause the software or the controller of the breast pump to output an alarm. The alarm may in turn trigger a measure to increase milk yield, e.g. manual or automatic adaptation of at least one operational parameter of the breast pump or other measures as required and/or desired. The alarm may be generated as soon as the tolerance value is overcome or with a delay, e.g. one day, in case the measured milk yield stays below the tolerance value.

FIG. 4 exemplifies some parameters of a pumping cycle with multiple sucking pattern. In FIG. 4, RS identifies the release suction, which is the highest pressure value within one cycle C i.e. the lowest suction pressure of the cycle C. PS identifies the pull suction, which is the lowest pressure value within one cycle C i.e. the highest suction pressure.

In the example of FIG. 4, the negative pressure within the breast shield will not become zero. The pump is adapted to maintain a baseline vacuum BV that can be about −10 kpa. A breast pump adapted to maintain such baseline vacuum is e.g. described in US2008077082 A1 or EP 4 015 012 A1.

What is claimed is:

1. A method for managing breast milk expression comprising:
providing an expected milk yield over time;
expressing milk and measuring the expressed milk yield at a given time;
comparing the measured milk yield at the given time with the expected milk yield at the given time;
generating an alarm if the measured milk yield is lower than the expected yield.

2. The method of claim 1, wherein the expected milk yield is provided with reference to a baby birth, and a measured milk yield is measured with reference to the baby birth.

3. The method of claim 1, wherein the alarm is generated with a delay with respect to the given time.

4. The method of claim 1, wherein the expected milk yield has a tolerance value, wherein the alarm is generated when the measured milk yield is lower than the tolerance value.

5. The method of claim 4, wherein the tolerance value is 10% or 5% or 3% less than the expected milk yield at a prefixed time or 10% or 5% or 3% less than the expected milk yield at an average value at different prefixed times.

6. The method of claim 5, wherein the prefixed time is day 6 and/or day 7 after baby birth.

7. The method of claim 1, wherein the alarm triggers at least one measure, which measure has the potential to increase milk yield.

8. The method of claim 1, wherein the method is implemented by a software executed by a processor of a smartphone or tablet or computer.

9. The method of claim 1, wherein the method is implemented by a software executed by a processor of a controller of a breast pump.

10. The method as defined in claim 8, wherein the software outputs information on the daily yield over time together with a path of expected yield over a time.

11. The method as defined in claim 9, wherein the alarm triggers the controller to change at least one operational parameter of the breast pump for increasing yield.

12. The method as defined in claim 11, wherein the controller changes at least one of the following operational parameters of the pumping operation of the breast pump to increase yield: cycle rate, vacuum level, pull suction and/or release suction, and/or activate the breast pump to apply a massage to the breast;
wherein vacuum level is the average vacuum provided by one cycle, wherein pull suction is the suction of a cycle with the lowest pressure and wherein release suction is the suction of a cycle with the highest pressure.

13. The method as defined in claim 12, wherein within a breast pumping session the controller controls a pattern of sequences, which pattern comprises at least one stimulation sequence for stimulating the breast having a rapid cycle rate and providing a moderate vacuum level and at least one expression sequence having a lower cycle rate and providing a stronger vacuum level, wherein the lower cycle rate is lower than the rapid cycle rate and in which method and in case of a yield deficit the controller changes at least one of the cycle rate, the vacuum level and the duration of at least one of the sequences.

14. A software comprising instructions that, when executed by at least one programmable processor, cause the at least one programmable processor to implement the method of claim 1.

15. A breast pump comprising an aggregate and a controller, the controller having at least one programmable processor, wherein when the programmable processor executes the software according to claim 14.
